(19) United States
(12) Patent Application Publication
(10) Pub. No.: US 2009/0317840 A1
(43) Pub. Date: Dec. 24, 2009

(54) AUTOMATED DETERMINATION OF CHLAMYDIA INFECTION-FORMING UNITS

(76) Inventor: Jon H. Heinrichs, Doylestown, PA (US)

Correspondence Address: MERCK AND CO., INC, P O BOX 2000, RAHWAY, NJ, 07065-0907, US

(21) Appl. No.: 12/309,668

(22) PCT Filed: Jul. 23, 2007
(86) PCT No.: PCT/US07/16565

§ 371 Date: Jan. 23, 2009

Related U.S. Application Data
(60) Provisional application No. 60/833,637, filed on Jul. 27, 2006.

(51) Int. Cl. G01N 33/571

(52) U.S. Cl. 435/7.36

(57) ABSTRACT

The present features an automated method of measuring Chlamydia infection forming units. The method employs a polypeptide recognizing Chlamydia infected inclusion body components. The polypeptide specifically binds to either, or both, a Chlamydia elementary body or reticulate body. Polypeptide binding is detected using a colorimetric indicator and an automated detector.

### CROSS-REFERENCE TO RELATED APPLICATIONS

This application claims benefit of U.S. Provisional Application No. 60/833,637, filed Jul. 27, 2006, which is hereby incorporated by reference herein.

### BACKGROUND OF THE INVENTION

The references cited in the present application are not admitted to be prior art to the claimed invention.

Chlamydia trachomatis is an obligate intracellular pathogen responsible for ocular trachoma and several sexually transmitted diseases. C. trachomatis serovars A, B, Ba, and C are responsible for ocular trachoma which can cause conjunctivitis, conjunctival scarring and corneal scarring. C. trachomatis serovars D, Da, E, F, G, H, I, Ia, J, Ja and K are responsible for oculogenital disease which can cause cervicitis, urethritis, endometritis, pelvic inflammatory disease, tubal infertility, ectopic pregnancy, neonatal conjunctivitis and infant pneumonia. Chlamydia trachomatis serovars L1, L2 and L3 are responsible for lymphogranuloma venereum which can cause submocosa and lymph-node invasion, with necrotizing granulomas and fibrosis. (Reviewed in Brunham et al., Nature Reviews Immunology 5:149-161, 2005; Montoya, Chlamydia, p. 694-702, In Wilson et al., Eds. Current Diagnosis & Treatment in Infectious Diseases, The McGraw-Hill Companies, Inc. 2001.)

During its development cycle C. trachomatis, like other Chlamydia, exists outside a host cell as a metabolically inactive infectious elementary body (EB). The elementary body is internalized by a host cell and surrounded by an endosomal membrane forming an inclusion body. Inside the inclusion body, the elementary body transforms into a metabolically active reticulate body (RB). The reticulate body divides by binary fusion. Within about 40-48 hours, the reticulate bodies transform back to elementary bodies. The produced elementary bodies are released by the host cell and can infect neighboring cells. (Id.)

### SUMMARY OF THE INVENTION

The present features an automated method of measuring Chlamydia infection forming units. The method employs a polypeptide recognizing Chlamydia infected inclusion body components. The polypeptide specifically binds to either, or both, a Chlamydia elementary body or reticulate body. Polypeptide binding is detected using a calorimetric indicator and an automated detector.

Thus, a first aspect of the present invention describes an automated method for measuring Chlamydia infection forming units. The method comprises the steps of: (a) incubating mammalian cells susceptible to Chlamydia infection in a cell culture with infectious Chlamydia under incubation conditions and for a sufficient time to allow formation of Chlamydia infected inclusion bodies; (b) fixing the cells; and (c) detecting Chlamydia infected inclusion body components as an indication of Chlamydia infection forming units using a polypeptide that specifically binds to Chlamydia infected inclusion body components, a calorimetric indicator, and an automated detector.

The colorimetric indicator produces a calorimetric signal indicating the presence of the detection polypeptide bound to Chlamydia infected inclusion body components. The calorimetric signal is detected by the automated detector.

Infectious Chlamydia refers to Chlamydia elementary bodies initially used in the method to infect host cells and produce inclusion bodies. The Chlamydia elementary bodies can be from different strains of Chlamydia such as C. trachomatis, C. psittaci and C. pneumoniae.

Reference to an “automated” method indicates that the Chlamydia infection forming units are detected and quantitated by a device, rather than being manually detected and quantitated by a person.

A polypeptide specifically binding to Chlamydia infected inclusion body components, binds to Chlamydia infected inclusion body components to a significantly greater extent than to cell components not infected with Chlamydia. The significantly greater extent is sufficient to provide for detection of the inclusion bodies and takes into account background effects.

A colorimetric indicator is a substance that produces a colorimetric signal. The colorimetric signal is a color, or a change in color, which can be detected. The calorimetric signal can be directly produced by the calorimetric indicator. The colorimetric signal can also be produced by the colorimetric indicator causing an alteration of a second substance, where the alteration produces the signal.

Reference to open-ended terms such as “comprises” allows for additional elements or steps. Occasionally phrases such as “one or more” are used with or without open-ended terms to highlight the possibility of additional elements or steps.

Unless explicitly stated, reference to terms such as “a” or “an” is not limited to one. For example, “a cell” does not exclude “cells”. Occasionally phrases such as one or more are used to highlight the possible presence of a plurality.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIGS. 1A-1B illustrate Chlamydia infectious forming units (IFU) images and counts of C. trachomatis serovar D. FIG. 1A illustrates Hak cell monolayer infected with 4000 EB/well (average spot size=−2.8 to −2 Log Sq. mm), and the detection of 77 counts. Detected counts were circled based on predefined parameters. FIG. 1B illustrates uninfected controls treated with sucrose phosphate glutamic acid (SPG), where 0 counts were detected.

FIGS. 2A-2B illustrates Chlamydia IFU images and counts of C. trachomatis serovar L2 in Hak cells. FIG. 2A illustrates results with 12500 EB/well, where 300 counts were detected. FIG. 2B illustrates results with 6250 EB/well, where 130 counts were detected.

FIG. 3 illustrates results from a C. trachomatis serovar L2 titration on 96 well plates with different host strains.

### DETAILED DESCRIPTION OF THE INVENTION

The present invention features an automated method of measuring Chlamydia infection forming units using a calorimetric system. The use of an automated calorimetric method provides several advantages to manual fluorescent microscopic methods. Advantages include less labor, adaptability to high throughput, and less investigator bias.

The automated methods can be used in different settings such as the quantification of elementary bodies recovered from clinical samples (such as urethral, genital, rectal, ocular or other site swabs), determination of elementary bodies purified from cell culture, evaluating potency of antibody responses to vaccines, evaluating vaccine efficacy, evaluating antibody inhibitors of Chlamydia infection, evaluating small molecule inhibitors of Chlamydia infection, and evaluating the effect of antibiotics. Evaluating potency of antibody responses to vaccines can be performed by evaluating the neutralizing activity of sera or purified antibody from an animal or patient vaccinated with a Chlamydia antigen.

Vaccine, antibody, and small molecule efficacy can be evaluated in animal models such as models of genital tract infection in the mouse, guinea pig or non-human primate, of the respiratory tract in mice, or of the conjunctiva in guinea pigs or non-human primates. (See, for example, Rank, R. G., 1999. Models of Immunity, p. 239-29. In R. S. Stephens (ed.), Chlamydia: Intracellular Biology, Pathogenesis and Immunity, American Society for Microbiology, Washington, D.C.) Vaccine efficacy can be evaluated in animals immunized with a vaccine and infected with Chlamydia, and using material containing, or expected to contain, elementary bodies from the animal. Similarly, antibody and small molecule efficacy can be evaluated by infecting the animal with Chlamydia, treating with the antibody or small molecule, and using material containing, or expected to contain, elementary bodies from the animal.

The overall method involves the following steps: (I) Chlamydia incubation; (II) Fixing; and (III) Detection. Additional steps can also be performed to enhance the assay. A preferred additional step is a washing step that occurs after fixing.

### I. Chlamydia Incubation

Chlamydia incubation provides host cells with infectious Chlamydia under conditions, and for a sufficient time, to allow formation of Chlamydia infected inclusion bodies. The infectious form of Chlamydia is an elementary body. Different Chlamydia strains and Chlamydia serovars can be employed.

Chlamydia elementary bodies can be obtained from different types of samples including cell culture and clinical samples. Techniques for obtaining and purifying elementary bodies are well known art. (For example see, Kawa et al. Vaccine 22:4282-4286, 2004.)

Suitable mammalian cells and growth conditions are selected taking into account the employed Chlamydia. Host strains and conditions for Chlamydia infection are well-known in the art. (See, for example, Schachter, J., and W. E. Stamm. 1999. Chlamydia, p. 795-806. In Murray, P. R., Baron, E. J., Pfaller, M. A., Tenover, F. C., and R. H. Yolken (eds.), Manual of Clinical Microbiology, 7^{th} edition. American Society for Microbiology, Washington, D.C.)

In an embodiment, the method is performed using C. trachomatis elementary bodies. C. trachomatis can be cultured in different types of cells including L292, McCoy, Hela, and Hak cells.

In another embodiment, the method is performed using C. pneumoniae. C. pneumoniae can be cultured in different types of cells including Hep-2, Hela and Hak. A preferred cell for C. pneumoniae is Hela.

The incubation time allowing for Chlamydia infected inclusion bodies formation varies a bit depending on the cell type and growth conditions. In an embodiment, the incubation time is for at least about 20 hours. Preferably, maximum incubation time occurs prior to significant cell lysis due to Chlamydia infection. In different embodiments, the minimum incubation time is about 20 hours, about 30 hours, about 40 hours or about 48 hours and the maximum time, which may be used with any of the minimum incubation times, is about 60 hours or about 72 hours.

In an embodiment of the present invention, the infected host cells are cultured under conditions preferentially inhibiting host cell replication (over Chlamydia replication). For example, the host cells can be cultured in the presence of a sufficient amount of cycloheximide to inhibit host cell growth. Inhibiting host cell growth facilitates inclusion body detection by decreasing the dilution effect of host cell growth.

Preferably, the Chlamydia is grown on a solid surface.

### II. Fixing

Fixing refers to the process of permeablizing a cell and adhering cellular components to a surface. In the present method, fixing is performed to permeablize the host cells and adhere Chlamydia elementary bodies and/or reticulate bodies, present in an inclusion body, to a surface. Fixing agents are well known in the art. (See, for example, Murray et al., 1994. Determinative and Cytological Light Microscopy, p. 21-41. In Gerhardt, P., Murray, R. G. E., Wood, W. A., and N. R. Krieg (eds.), Methods for General and Molecular Bacteriology, American Society for Microbiology, Washington, D.C.) Examples of fixing agents include ethanol, methanol, isopropanol, acetone, formalin, formaldehyde, and glutaraldehyde.

### III. Inclusion Body Component Detection

Chlamydia infected inclusion body components are measured using a detection polypeptide specific for a Chlamydia elementary body and/or reticulate body. Detection polypeptide binding is evaluated using a colorimetric indicator in conjunction with an automated detector.

### A. Detection Polypeptide

The detection polypeptide is specific for a Chlamydia elementary body and/or reticulate body. Reference to “polypeptide” indicates a contiguous amino acid sequence and does not provide a minimum or maximum size limitation. One or more amino acids present in the polypeptide may contain a post-translational modification, such as glycosylation or disulfide bond formation. Preferably, the polypeptide contains an antibody variable region that recognizes an elementary body and/or reticulate body epitope.

An antibody variable region contains three complementary determining regions interspaced onto a framework. The complementary determining regions are primarily responsible for recognizing a particular epitope. Antibody variable regions can be present in different types of polypeptides such as single-chain antibodies, a complete antibody, an antibody fragment, and derivatives thereof.

In different embodiments the detection polypeptide is provided as a monoclonal antibody or a polyclonal antibody. Reference to a “monoclonal antibody” indicates a collection of antibodies having the same, or substantially the same, complementary determining regions and binding specificity. The variation in the monoclonal antibodies is that which would occur if the antibodies were produced from the same construct(s).

Monoclonal antibodies can be produced, for example, from a hybridoma and from a recombinant cell containing one or more recombinant genes encoding the antibody. The antibody may be encoded by more than one recombinant gene where, for example, one gene encodes the heavy chain and one gene encodes the light chain.

Antibody fragments containing an antibody variable region include Fv, Fab, and F(ab′)₂ regions. Each Fab region contains a light chain containing a variable region and a constant region, and a heavy chain region containing a variable region and a constant region. The light and heavy chains are joined by disulfide bonding through constant regions. The light and heavy chain variable regions of a Fab region provide for an Fv region that participates in antigen binding.

The antibody variable region can also be part of polypeptide containing variable regions such as a single chain antibody and a minibody. A single chain antibody contains a light and a heavy variable region joined together by a linker. A minibody is a single chain-CH3 fusion protein that self assembles into a bivalent dimer of about 80 kDa.

Antibodies recognizing a Chlamydia elementary body and/or reticulate body are known in the art and are commercially available. For example, Chlamydia trachomatis LPS mAb, (mouse IgG2a, Catalog #15174), recognizes serovar A, B, Ba, C, D, E, F, G, H, I, J, K, L1, L2, L3 (QED Bioscience, Inc., San Diego, Calif.).

Polypeptides recognizing an elementary body and/or reticulate body can also be produced using techniques such as those producing a single-chain antibody, a complete antibody, or an antibody fragment. Examples of such techniques include the use of phage display technology, isolation of sera from animals infected with Chlamydia or a Chlamydia subunit, and hybridoma production from an animal infected with Chlamydia or a Chlamydia subunit.

### B. Colorimetric Indicator

The colorimetric indicator produces a calorimetric signal indicating the presence of detection polypeptides bound to Chlamydia elementary bodies and/or reticulate bodies. Signal production from bound detection polypeptide allows for the visualization of Chlamydia infected inclusion bodies.

The calorimetric indicator either directly provides a calorimetric signal or acts on another substance to provide the signal. Directly providing a colorimetric signal can be achieved using different types of dyes such as Indigo and Texas Red.

A substance acting on another substance can be an enzyme that cleaves a substrate to produce a calorimetric signal. Enzyme substrate combinations that could be used to produce a colorimetric signal are well known in the art. Examples of such combinations include horseradish peroxidase used with DAB/H₂O₂ (“DAB” refers to 3,3″-diaminobenzidine); alkaline phosphatase used with NBT/BCIP (“NBT/BCIP” refers to nitro-blue tetrazolium chloride/5-bromo-4-chloro-3-indolyl-phosphate); glucose oxidase used with TNBT/PMS (“TNBT” refers to tetranitroblue tetrazolium); and β-galactosidase used with Nap-Gal/hexazonium-p-rosaniline. (See, for example Savage et al., Avidin-Biotin Chemistry: A handbook, p. 145-190, developed by Pierce, available in PDF format http://www.piercenet.com/Objects/View.cfm?type=Page&ID=E82B5AEB-A192-4548-83BF-9229E3397C6C.)

Techniques for conjugating a dye or an enzyme to a polypeptide are well-known in the art. (See, for example, Harlow, E. and D. Lane. 1988. Antibodies: A Laboratory Manual. Cold Spring Harbor Laboratory, Cold Spring Harbor, N.Y.)

The detection polypeptide could also be detected with a colorimetric indicator conjugated to a substance that specifically binds the detection polypeptide. Specific binding to the detection polypeptide is with respect to other substances present, and is sufficient to distinguish binding to the detection polypeptide from other substances present. Specific binding can be achieved in different ways, such as:

1) providing the detection polypeptide with a ligand, and using a protein that recognizes the ligand and is conjugated to a calorimetric indicator, alternatively the detection polypeptide may contain the protein and it is bound by a ligand conjugated to the colorimetric indicator;

2) using a secondary polypeptide to specifically bind the detection polypeptide, where the secondary polypeptide is conjugated to a colorimetric indicator; and

3) using a secondary polypeptide to specifically bind the detection polypeptide, where the secondary polypeptide has a ligand that will bind a protein conjugated to a colorimetric indicator, alternatively the secondary polypeptide may contain the protein and is bound by a ligand conjugated to the colorimetric indicator.

Examples of protein/ligand combinations include avidin/biotin, streptavidin/biotin, and protein A/immunoglobulin Fc.

Preferably, the secondary polypeptide contains an antibody variable region. As noted in III.A. Detection Polypeptides, supra, antibody variable regions can be present in different types of polypeptides such as single-chain antibodies, a complete antibody, an antibody fragment, and derivatives thereof.

### C. Automated Detector

The automated detector is an apparatus that detects the presence of the colorimetric signal and provides a readout related to the number of discrete colorimetric signals in a particular area. The particular areas that are evaluated can be preset by an apparatus user. Preferably, when the assay is performed in a multi-well plate, whole wells are evaluated rather than well fractions.

Detection can be achieved by photographic means such as through the use of a camera. Preferably, a charge-coupled device (CCD) camera is employed. A CCD camera uses a silicon wafer to receive incoming light. The silicon wafer is a solid-state electronic component segmented into an array of individual light-sensitive cells, also referred to as “pixels”. Each pixel is an element of the whole picture. Digitized images can be analyzed to identify changes in color density due to the colorimetric signals.

The readout related to the number of discrete calorimetric signals can be achieved using appropriate software. The data can be presented in different formats such as an Excel sheet.

Preferred automated detectors are those currently in use for Elispot assays. Examples of these include an ImmunoSpot Image Analyzer such as the Series 3A, 3B or 3C Analyzer (CTL) and the BIOREADER 3000/4000 PRO-X (BIO-SYS GmbH). Hesse et al., The Journal or Immunology 167:1353-1361, 2001 and, Zielinska et al., Virology Journal 2:1-5, 2005 provide examples of using an ImmunoSpot Image Analyzer.

### EXAMPLES

Examples are provided below to further illustrate different features of the present invention. The examples also illustrate useful methodology for practicing the invention. These examples do not limit the claimed invention.

### Example 1

### Determining Inclusion Forming Units in Hak Cells

This example illustrates the detection of inclusion forming units in Hak cells using an antibody directed to C. trachomatis lipopolysaccharide (LPS) as a detection probe, that is bound by a second antibody conjugated to biotin, and detected using Streptavidin-AP conjugate and NBT/BCIP.

### Material and Reagents:

1. 96-well tissue culture plate, white (Nunclon # 136102).
2. Purified Chlamydia EB preparation (see, for example, Kawa, D. E. and R. S. Stephens. J. Immunology. 168:5184-5191, 2002)
3. Hak cell, ATCC#CCL-15

### 4. Methanol, Fisher #A412-4

5. Anti-Chlamydia genus LPS mAb, mouse QED Bioscience, Inc. (catalog #15174)
6. Goat anti-mouse IgG (γ-chain specific), biotin conjugate, SBT #1030-08
7. Streptavidin-AP conjugate, Pierce (catalog #21323)
8. NBT/BCIP, 1-step AP substrate, Pierce, # 34042

### Media and Solutions:

1. Hak Medium: Basal Medium Eagle, 90%, Invitrogen # 21010-046 Fetal bovine serum, 10%, heat inactivated, Hyclone, # SH30070.03 L-glutamine, 2 mM, Gibco#25030-081 Vancomycin, 50 ug/ml, Sigma # V-2002
2. Sucrose Phosphate Glutamic acid (SPG): 75 g Sucrose+0.5 g KH₂PO₄+1.2 g Na₂HPO₄+0.72 g L-glutamic acid, 1 L sterile ddH₂O. Adjust pH to 7.4-7.6 with 2 N NaOH.
3. Cycloheximide 200 ug/ml stock, used at 1:100 dilutions, 2 ug/ml in medium final.
4. HBSS, 1×, Cellgro #21-020-CV
5. PBS
6. Blocking buffer: 10% FBS, 90% PBS, 0.05% Tween-20
7. Plate wash solution: 0.05% Tween-20 in PBS

### Equipment and Software:

1. Immunospot counter (CTL)

### 2. ImmunoSpot-3 (CTL)

3. AMP plate washer

### Growth Conditions

1. Seed Hak cells at 2-4×10⁴ cells/well into a 96well plate and incubate the plate overnight at 37° C. and 5% CO₂ prior to infection with C. trachomatis EBs.
2. Aspirate media and rinse cells with HBSS once.
3. Prepare infectious EB solution: dilute bacteria to desired concentration. For neutralization assays, preincubate mixture of bacteria and antibody in SPG.
4. Add the infectious EB solution to washed Hak monolayers.
5. Incubate the plate at 37° C. for 2 hours on a plate rocker.
6. Wash the plate twice with HBSS.
7. Add complete culture media containing 2 ug/ml Cycloheximide, 200 ul/well.
8. Incubate for 44-48 hours at 37° C. and 5% CO₂.

### Chlamydia IFU Staining:

1. Wash the plate one time with HBSS, 200 ul/well following the incubation for growth of inclusion bodies. Fix the plate with absolute methanol, 100 ul/well and sit the plate at room temperature for 20 minutes.
2. Wash the plate twice with PBS, 200 ul/well following the fixation.
3. Block the plate with blocking buffer, 100 ul/well and incubate for 2 hours at room temperature (in some instances the plates are placed on a rotator with slow rotating rate).
4. Wash the plate three times with PBS/Tween-20.
5. Prepare mAb to Chlamydia genus LPS in blocking buffer at 0.1 ug/ml (or 1:10000), and add to the plates 50 ul/well. Cover the lids and incubate the plate at room temperature for 2 hours (in some instances the plates are placed on a rotator with a slow rotating rate).
6. Wash the plate as in step 4.
7. Prepare goat anti-mouse IgG-biotin conjugate at 1 ug/ml (or 1:1000 dilution) in blocking buffer. Add the 2^{nd} Ab to the plate, 50 ul/well and cover the lid. Incubate the plate at room temperature for 2 hours (in some instances the plates are placed on a rotator with a slow rotating rate).
8. Wash the plate as in step 4.
9. Prepare Streptavidin-AP 1:3000 dilution in blocking buffer. Add the conjugate to the plate, 50 ul/well and cover the lid. Incubate the plate at room temperature for 1 hour (in some instances the plates are placed on a rotator with a slow rotating rate).
10. Wash the plate 3× with PBS/Tween-20 and 3× with PBS.
11. Add AP substrate NBT/BCIP 50 ul/well and incubate for 2-3 minutes. Wash the plate with tap water and dry under airflow.

### Chlamydia IFU Enumeration:

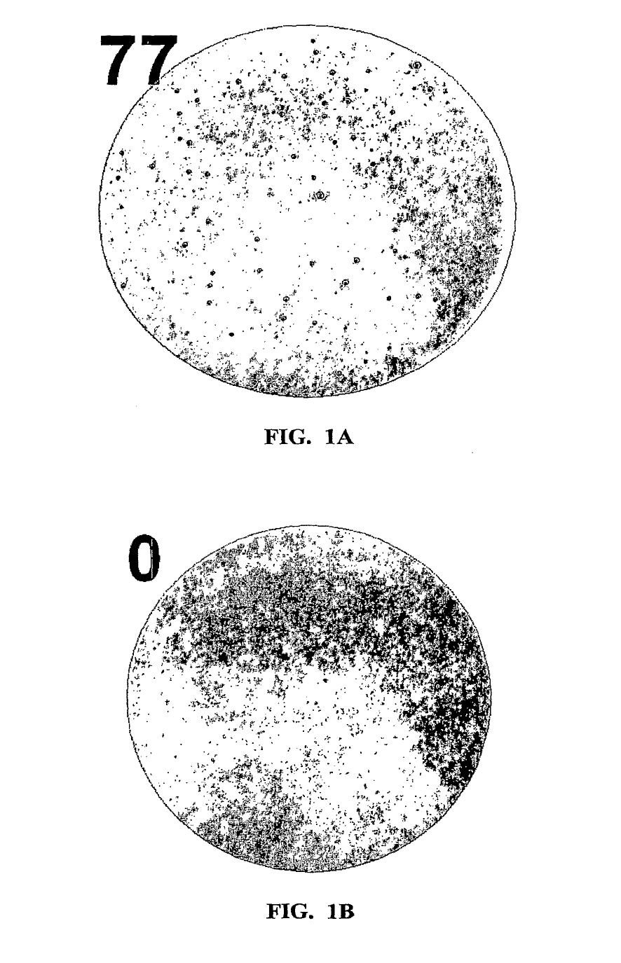

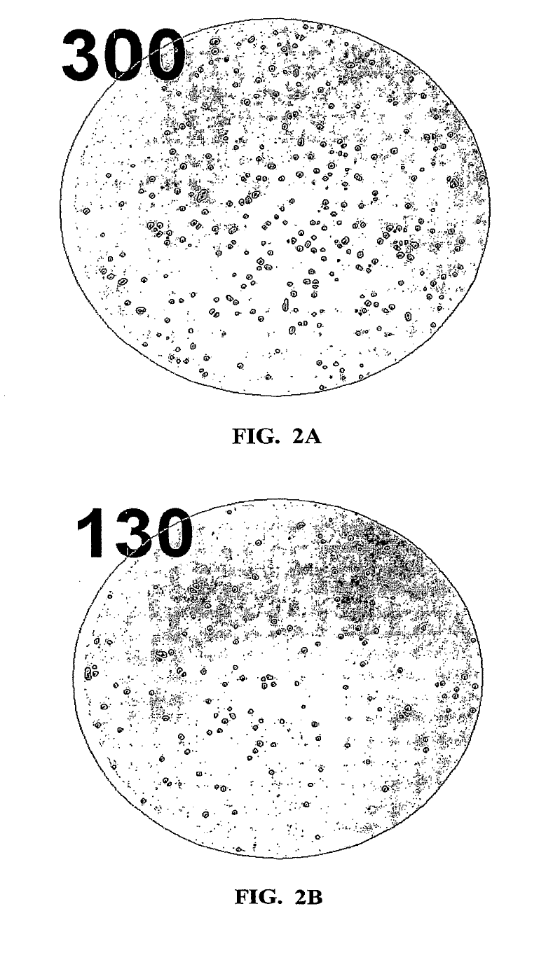

Chlamydia IFUs are imaged and counted using an Immunospot Counter. The plate is scanned. The instrument automatically acquires and stores raw images from each plate well and uses a set of macros from Optimas software package to perform IFU counts based on user-defined criteria. FIGS. 1A and 1B illustrate C. trachomatis serovar D IFU images and counts. FIGS. 2A and 2B illustrate C. trachomatis serovar L2 IFU images and counts. Data from the Immunospot counter can also be exported to, for example, an Excel worksheet for analysis and presented in a table format.

### Example 2

### Determining Inclusion Forming Units in Different Cell Types

This example illustrates the use of different host strains. L929 (ATCC # CCL-1), McCoy (ATCC # CRL 1696), HeLa (ATCC # CCL-2.1), and Hak cells were used. The material and techniques described in Example 1 were employed with the follow changes in medium for HeLa, L929, and McCoy:

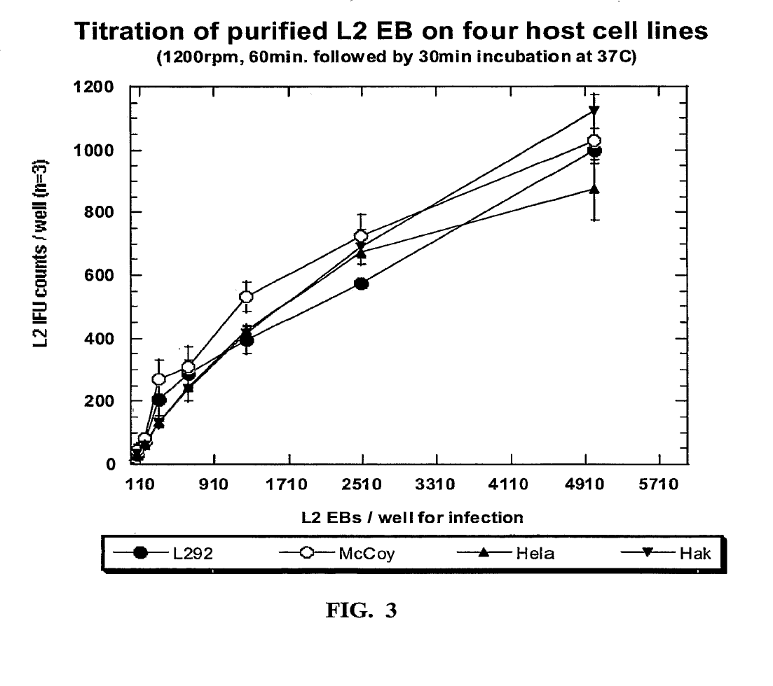

ATCC Eagle's Minimum Essential Medium (EMEM—ATCC # 30-2003); 10% FBS; 50 μg/ml Vancomycin; and 10 μg/ml Gentamicin. The results are show in FIG. 3.

### Example 3

### Neutralizing Activity of Anti-Serum from SerovarD EB Immunized Mice

Neutralizing activity of various serum samples was tested essentially as described by Byrne et al., J. Infect. Dis. 168:415-420, 1993, except that following growth of Chlamydia in Hak cells, detection of inclusion bodies was performed as described above beginning at step 1 of Chlamydia IFU staining.

Results from a determination of neutralizing activity of sera obtained from immunized mice using an automated assay are illustrated in Tables 1 and 2. Table 1 shows the number of inclusion spots obtained from EB-infected Hak cells following preincubation with various concentrations of test antisera. Samples were tested in duplicate wells except controls which were tested in replicates of eight wells each. Table 2 shows mean counts from replicate wells and percent neutralization of EB infectivity compared to no serum control.

TABLE 1

| Serum dilution | D-EB | Serum 1 | | Serum 2 | | Serum 3 | | Serum 4 | | Serum 5 | | Controls | | |
|---|---|---|---|---|---|---|---|---|---|---|---|---|---|---|
| 10 | A | 20 | 27 | 8 | 5 | 5 | 2 | 7 | 3 | 8 | 9 | 54 | 80 | |
| 30 | B | 36 | 56 | 6 | 4 | 10 | 8 | 4 | 4 | 12 | 6 | 76 | 89 | <-No serum |
| 90 | C | 68 | 61 | 1 | 2 | 4 | 8 | 3 | 10 | 3 | 9 | 71 | 82 | control |
| 270 | D | 101 | 68 | 18 | 11 | 2 | 11 | 2 | 3 | 3 | 7 | 95 | 74 | |
| 810 | E | 91 | 95 | 52 | 72 | 28 | 20 | 8 | 12 | 12 | 12 | 0 | 0 | |
| 2430 | F | 78 | 99 | 83 | 73 | 52 | 69 | 43 | 32 | 31 | 38 | 1 | 1 | <-SPG only |
| 7290 | G | 108 | 93 | 108 | 82 | 81 | 77 | 51 | 72 | 71 | 80 | 0 | 0 | no EBs or sera |
| 21870 | H | 86 | 70 | 71 | 76 | 75 | 73 | 69 | 61 | 69 | 64 | 0 | 0 | |

TABLE 2

| | | Serum 1 | | Serum 2 | | Serum 3 | | Serum 4 | | Serum 5 | | Controls | |
|---|---|---|---|---|---|---|---|---|---|---|---|---|---|
| Serum | | | % | | % | | % | | % | | % | | % |
| dilution | | Mean | Neut. | Mean | Neut. | Mean | Neut. | Mean | Neut. | Mean | Neut. | Mean | Neut. |
| 10 | A | 24 | 70 | 7 | 92 | 4 | 96 | 5 | 94 | 9 | 89 | 78 | 0 |
| 30 | B | 46 | 41 | 5 | 94 | 9 | 89 | 4 | 95 | 9 | 89 | | |
| 90 | C | 65 | 17 | 2 | 98 | 6 | 93 | 7 | 92 | 6 | 93 | | |
| 270 | D | 85 | 0 | 15 | 82 | 7 | 92 | 3 | 97 | 5 | 94 | | |
| 810 | E | 93 | 0 | 62 | 20 | 24 | 69 | 10 | 87 | 12 | 85 | 0 | |
| 2430 | F | 89 | 0 | 78 | 0 | 61 | 22 | 38 | 52 | 35 | 56 | | |
| 7290 | G | 101 | 0 | 95 | 0 | 79 | −2 | 62 | 21 | 76 | 3 | | |
| 21870 | H | 78 | 0 | 74 | 5 | 74 | 5 | 65 | 16 | 67 | 14 | | |

### Example 4

### C. pneumonia EB Titration on Hak, Hela and Hep-2 Monolayers

Inclusion-forming units were also enumerated following infection of Hak, HeLa or Hep-2 cell lines with Chlamydia pneumoniae strain TWAR essentially as described for C. trachomatis with the following exceptions:

1. Plates were centrifuged at 1,200 rpm for 60 minutes after addition of strain TWAR to cell monolayers.

2. Following centrifugation, plates were incubated at 37° C. for 30 minutes in a 5% CO₂ incubator.

3. Hep-2 cells were grown in Iscove's Modified Dulbecco's Medium with 10% FBS, 2 mM L-glutamine, 50 μg/ml Vancomycin and 10 μg/ml Gentamicin

Results from a C. pneumoniae infection study are shown in Table 3. Decreasing concentrations of EBs, from 12,500 to 6 (as determined by manual microscopic counting of IFUs) was added to cell monolayers of Hak, HeLa or Hep-2 cells. Inclusion forming spots were determined as described above. Spots could not be quantified following infection of Hep-2 cells due to a high background signal. IFUs produced by strain TWAR were generally smaller than those produced by C. trachomatis.

TABLE 3
Automated spot counts

| | | Hak | HeLa | Hep-2 |
|---|---|---|---|---|
| TWAR EB/well | | Average | Average | Average |
| 12500 | A | 274 | 910 | 1477 |
| 4167 | B | 142 | 574 | 1279 |
| 1389 | C | 73 | 340 | 1261 |
| 463 | D | 27 | 126 | 1249 |
| 154 | E | 12 | 50 | 1223 |
| 51 | F | 4 | 27 | 1212 |
| 17 | G | 5 | 24 | 1201 |
| 6 | H | 1 | 29 | 1456 |

Other embodiments are within the following claims. While several embodiments have been shown and described, various modifications may be made without departing from the spirit and scope of the present invention.

What is claimed is:

1. An automated method for measuring Chlamydia infection forming units comprising the steps of:
(a) incubating mammalian cells susceptible to Chlamydia infection in a cell culture with infectious Chlamydia under incubation conditions and for a sufficient time to allow formation of Chlamydia infected inclusion bodies;
(b) fixing said cells; and
(c) measuring Chlamydia infected inclusion body components as an indication of said Chlamydia infection forming units using a polypeptide that specifically binds to said Chlamydia infected inclusion body components, a colorimetric indicator, and an automated detector;
wherein said colorimetric indicator produces a colorimetric signal indicating the presence of said polypeptide bound to said Chlamydia infected inclusion body components, and said colorimetric signal is detected by said automated detector.

2. The method of claim 1, wherein said automated detector contains a charge-coupled device camera and the Chlamydia is grown on a solid surface.

3. The method of claim 2, wherein said method is used to detect Chlamydia trachomatis or Chlamydia pneumoniae infection forming units.

4. The method of claim 3, wherein said step (a) is performed under conditions preferentially inhibiting mammalian cell replication.

5. The method of claim 4, wherein said conditions preferentially inhibiting mammalian cell replication employ a sufficient amount of cycloheximide to inhibit mammalian cell replication.

6. The method of claim 5, wherein said polypeptide is a monoclonal antibody that binds to either, or both, reticulate bodies or elementary bodies, that are present in said Chlamydia infected inclusion bodies

7. The method of claim 6, wherein cell debris is removed between said step (b) and said step (c).

8. The method of claim 7, wherein said sufficient time is at least about 20 hours.

9. The method of claim 8, wherein said monoclonal antibody comprises said calorimetric indicator and said colorimetric indicator is an enzyme that cleaves a colorimetric substrate to produce said calorimetric signal.

10. The method of claim 8, wherein a secondary antibody is used to bind said monoclonal antibody, said secondary antibody comprises said colorimetric indicator, and said colorimetric indicator is an enzyme that cleaves a colorimetric substrate to produce said calorimetric signal.

11. The method of claim 8, wherein a secondary antibody is used to bind said monoclonal antibody, said secondary antibody comprising a ligand, and said colorimetric indicator is conjugated to an enzyme that binds said ligand.

12. The method of claim 11, wherein said calorimetric indicator is an enzyme that cleaves a colorimetric substrate to produce said calorimetric signal.

13. The method of claim 12, wherein said secondary antibody is biotinylated, said colorimetric indicator is alkaline phosphatase, which is conjugated to streptavidin, and said colorimetric signal is produced from a colorimetric alkaline phosphatase substrate.

14. The method of claim 13, wherein said substrate is nitro-blue tetrazolium chloride/5-bromo-4-chloro-3-indolyl-phosphate.

15. The method of claim 8, wherein said sufficient time is about 48 to about 72 hours.
